(19) United States
(12) Patent Application Publication
(10) Pub. No.: US 2026/0334875 A1
(43) Pub. Date: Oct. 29, 2026

(54) WEARABLE-COMPATIBLE BIOMETRIC BRIDGE AND RECHARGING INTERFACE

(71) Applicant: Mark II Applied Technologies, LLC, Johns Creek, GA (US)

(72) Inventor: Eric Paul Guyer, Johns Creek, GA (US)

(73) Assignee: Mark II Applied Technologies, LLC, Johns Creek, GA (US)

(21) Appl. No.: 19/187,406

(22) Filed: Apr. 23, 2025

(51) Int. Cl. A61B 5/00; A61B 5/01; A61B 5/024; A61B 5/053; A61B 5/145; A61B 5/1455

(52) U.S. Cl. CPC A61B 5/6801; A61B 5/01; A61B 5/02416; A61B 5/053; A61B 5/14532; A61B 5/1455; A61B 5/7455; A61B 2560/0214; A61B 2562/0271; A61B 2562/164; A61B 2562/227

(57) ABSTRACT

The present invention relates to a biometric bridge system configured to be positioned between a wearable device and the skin of a user. The system comprises an enclosure housing a controller, rechargeable power supply, and biometric sensor interfaces. The top surface of the biometric bridge couples with the wearable device, while the bottom surface conforms to the user's skin, enabling pass-through sensing, relay sensing, and enhanced sensing modes. The enclosure features dual-surface alignment structures that allow the biometric bridge to mate with both the wearable device and a charging base, enabling simultaneous recharging. Optionally, a thumb tab can extend from the enclosure to aid ergonomic placement and removal. The controller is web-enabled and may transmit biometric data wirelessly to external computing devices or remote servers. The system supports expanded sensing functionality, modular integration, and seamless operation without disrupting the wearable device's form factor.

### TECHNICAL FIELD OF THE INVENTION

The present invention relates generally to wearable electronics and health monitoring systems, and more particularly to a modular interface system configured to be positioned between a wearable device and the skin of a user. The invention pertains to systems and methods for maintaining and augmenting biometric sensing capabilities, providing on-body and off-body charging support, and enabling data communication between a wearable device and external computing or cloud-based resources. The invention further relates to the design of interoperable components for use with smartwatches, fitness trackers, and other body-worn electronic devices, including systems incorporating Internet of Things (IoT) communication, ergonomic enclosures, and sensor relay or enhancement technologies.

### BACKGROUND OF THE INVENTION

Before this invention, users of wearable electronic devices such as smartwatches, fitness bands, and biometric trackers faced significant limitations in maintaining uninterrupted sensing and monitoring throughout the day and night. These devices often relied on direct skin contact for accurate biometric data collection, yet their designs required removal from the body for charging or being tethered to a cord for wired-charging. As a result, continuous monitoring—particularly during sleep or during long-duration activities—was difficult to achieve without data gaps.

Furthermore, wearable devices have historically offered a fixed set of sensing capabilities that cannot be easily modified or enhanced after purchase. Users seeking advanced biometric insights, such as electrocardiogram (ECG) readings, non-invasive glucose levels, or more precise skin temperature data, have had to rely on separate devices or upgrade to new wearables altogether. These options not only introduce cost and inconvenience but also fail to offer a seamless user experience across devices.

Another persistent issue has been the disconnect between wearable device functionality and the device's power management architecture. Many wearables require proprietary or specialized charging accessories that force the user to remove the device from their wrist, interrupting both biometric measurement and user convenience. While some charging solutions exist, they often do not account for simultaneous device wear and sensor continuity, making the recharge process a disruptive event in the user's monitoring routine.

Integration with computing ecosystems—such as smartphones, tablets, desktop systems, and cloud-based services—has also been limited in many devices, particularly with regard to sensor data flexibility, user control, and real-time remote analysis. In systems where connectivity is available, it is often tightly controlled by the device's firmware, restricting integration with third-party health platforms or custom sensing workflows.

The present invention addresses these and other shortcomings by providing a novel interface system that operates between the wearable device and the skin of the user, offering mechanical compatibility, sensing continuity, and communication flexibility. For these reasons and shortcomings, as well as other reasons and shortcomings, there is a long-felt need that gives rise to the present invention.

### SUMMARY OF THE INVENTION

The shortcomings of the prior art are overcome and additional advantages are provided through the provision of a biometric bridge system configured to be positioned between a wearable device and the skin of a user. In an exemplary embodiment, the system includes a biometric bridge comprising an enclosure with a top surface and a bottom surface, and a coupling region on the top surface that is structured to engage a coupling area of the wearable device.

The system further comprises sensor interfaces on both sides of the enclosure—one between the biometric bridge and the wearable device, and one between the biometric bridge and the user. These sensor interfaces support multiple modes of biometric sensing and may include, for example, a wearable device sensor interface on the top surface and a skin-contact sensor interface on the bottom surface.

A controller is housed within the enclosure and operatively connected to one or both of the sensor interfaces. The controller is configured to enable at least one of three sensing modes:
Pass-through sensing, in which signals from the wearable device pass through the biometric bridge to the user's skin;
Relay sensing, in which the biometric bridge captures biometric signals and relays them to the wearable device; or
Enhanced sensing, in which the biometric bridge independently measures biometric parameters that the wearable device itself is not configured to detect.

This configuration allows the biometric bridge system to maintain or expand biometric monitoring without interrupting the physical coupling between the wearable device and the user, addressing prior limitations related to sensing gaps, fixed sensor capabilities, and hardware constraints.

Additional shortcomings of the prior art are overcome and additional advantages are provided through the provision of a biometric bridge system configured to be positioned between a wearable device and the skin of a user. In an exemplary embodiment, the system includes a biometric bridge comprising an enclosure having a top surface and a bottom surface, with structural features designed to integrate seamlessly with both the wearable device and the user's skin.

The top surface includes a concave coupling region configured to conform to the convex underside of the wearable device, ensuring a snug and stable mechanical fit. The bottom surface includes a convex skin-contact sensor interface that is shaped to match the curvature of the user's skin, thereby improving comfort, stability, and sensor contact. This same convex structure is further configured to align with a standard wearable device recharger mating surface, enabling the system to support dual-sided docking—mating with the wearable device on one side and the recharger on the other. In operation, a power cord can provide power to the wearable device recharger that can be used to recharge the wearable device, the biometric bridge, or the combination of the wearable device coupled to the biometric bridge 100 simultaneously.

To ensure precise alignment during use or charging, the system includes complementary alignment features—a ridge on one of the top or bottom surfaces and a corresponding groove on the opposite side. This mirrored geometry promotes both reliable coupling and reversibility.

A thumb tab is integrated into the enclosure to assist the user in positioning and removing the biometric bridge system quickly and accurately, even when the wearable device remains partially secured to the wrist.

A controller is disposed within the enclosure and is operatively coupled to one or more internal sensors and a rechargeable battery, enabling on-board biometric sensing, power management, and optional communication functions.

This embodiment provides a mechanically intelligent, ergonomic, and recharger-compatible platform that integrates easily with existing wearable devices while maintaining comfort, continuity, and modular functionality.

Additional shortcomings of the prior art are overcome and additional advantages are provided through the provision of a biometric bridge system configured to be positioned between a wearable device and the skin of a user. In an exemplary embodiment, the system includes a biometric bridge comprising an enclosure with a top surface and a bottom surface, and a coupling region that interfaces with the wearable device to support sensing, power, and communication functions.

A controller is housed within the enclosure and includes a microcontroller, memory, and a communication interface. The controller is electrically connected to a rechargeable battery, which is further configured to provide power to both the biometric bridge and the wearable device. The controller is also operatively connected to one or more biometric sensors disposed within or on the enclosure, enabling localized collection of user physiological data such as heart rate, temperature, or other metrics.

The communication interface of the controller is configured to transmit biometric sensor data to one or more of the following:
The wearable device, enabling direct integration and interpretation by the native device platform;
A computing device, such as a smartphone, tablet, or personal computer for real-time monitoring or visualization; or
A remote data processing resource over a global network, such as a cloud server or connected health platform, for long-term storage, analysis, or third-party access.

This embodiment enables the biometric bridge system to act as an intelligent, connected node within a broader biometric sensing and data infrastructure, offering real-time insight, cloud integration, and enhanced monitoring capabilities that extend beyond the native limitations of the wearable device.

Additional shortcomings of the prior art are overcome and additional advantages are provided through the provision of a biometric bridge system configured to be positioned between a wearable device and the skin of a user. In an exemplary embodiment, the system comprises a biometric bridge including an enclosure with a top surface and a bottom surface, and structural features that support both wearable integration and charging alignment.

The top surface includes a coupling region formed to mate with the coupling area of a wearable device. This coupling region comprises one or more first alignment structures selected from grooves, ridges, or dimples, which guide and secure the attachment of the wearable device to the biometric bridge. These features ensure reliable sensor alignment and mechanical stability during use.

The bottom surface includes a sensor interface that is configured to contact the skin of the user for biometric data acquisition. Also located on the bottom surface are second alignment structures that are complementary to the first alignment structures. These mirrored or inverted structures enable the biometric bridge to be repositioned and aligned with a wearable device recharging system when the wearable device is removed, allowing for continued or simultaneous charging.

A controller is disposed within the enclosure and operatively coupled to one or more biometric sensors and a rechargeable battery, enabling the system to power sensing operations, facilitate data communication, and maintain charge for both standalone and paired device use.

This configuration introduces a dual-orientation mechanical alignment system, enabling the same enclosure to mate with both the wearable device and the wearable device's charging platform—simplifying transitions between sensing and charging, and reducing user burden without sacrificing comfort or data continuity.

System and computer program products corresponding to the above-summarized methods are also described and claimed herein.

Additional features and advantages are realized through the techniques of the present invention. Other embodiments and aspects of the invention are described in detail herein and are considered a part of the claimed invention. For a better understanding of the invention with advantages and features, refer to the description and to the drawings.

### BRIEF DESCRIPTION OF THE FIGURES

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 illustrates one example of a biometric bridge system inserted between a wearable device and the skin of a user;

FIG. 2 illustrates one example of a biometric bridge system aligning and coupling with a wearable device, the top surface of the wearable device and the biometric bridge are visible;

FIG. 3 illustrates one example of a biometric bridge system aligning and coupling with a wearable device, the bottom surface of the wearable device and the biometric bridge are visible;

FIG. 4A illustrates one example of a top surface perspective view and a bottom surface perspective of the biometric bridge system coupled to the wearable device;

FIG. 4B illustrates one example of a biometric bridge configured as a disc shape and a ring shape;

FIG. 5 illustrates examples of a system block diagram of the controller;

FIG. 6 illustrates one example of a network diagram, including pass through sensing, relay sensing, and enhanced sensing between the wearable device and the biometric bridge system;

FIG. 7 illustrates one example of a top perspective view and a bottom perspective view of the coupled wearable device and biometric bridge system;

FIGS. 8-10 illustrate examples of top perspective views of the uncoupled wearable device and biometric bridge system;

FIGS. 11-13 illustrate examples of bottom perspective views the uncoupled wearable device and biometric bridge system;

FIG. 14 illustrates one example of a top view and a bottom view of the biometric bridge system;

FIG. 15 illustrates one example of a front view and a back view of the biometric bridge system;

FIG. 16 illustrates one example of a left view and a right view of the biometric bridge system; and

FIG. 17 illustrates examples of methods of using a biometric bridge system.

The detailed description explains the preferred embodiments of the invention, together with advantages and features, by way of example with reference to the drawings.

### DETAILED DESCRIPTION OF THE INVENTION

The present invention relates to a wearable interface system referred to herein as a biometric bridge system 100. This system is configured to be positioned between a wearable device 200, such as a smartwatch, and the skin of a user 302. The biometric bridge system 100 enables continuous biometric sensing, on-body charging of the wearable device, and multi-mode data communication without requiring removal of the wearable device or interruption of its intended functions. In doing so, the present invention addresses a number of long-standing technical limitations associated with wearable devices and their interaction with the human body.

In the present invention, the term “biometric bridge” is intended to mean a physical and functional interface system configured to be positioned between a wearable device and the skin of a user, wherein the system enables the acquisition, transmission, or augmentation of biometric data. The biometric bridge comprises structural elements that support direct skin contact, alignment with the sensing components of the wearable device, and housing for internal sensors and electronics. The biometric bridge may enable multiple sensing modes, including pass-through sensing, relay sensing, and enhanced sensing. It includes a controller, one or more biometric sensors, and a rechargeable battery. The rechargeable battery is configured not only to power the internal components of the biometric bridge itself but also to supply charging energy to the wearable device when coupled. The biometric bridge may further include communication interfaces for wired or wireless transmission of sensor data to a wearable device, a local computing device, or a remote data processing resource over a global network. The term also encompasses configurations that support dual-sided coupling with both a wearable device and a charging base 220, enabling uninterrupted sensing, modular upgradeability, and integrated power management within the wearable ecosystem.

In the present invention, the term “sensor interface” is intended to mean a physical and/or electrical structure that facilitates the transmission of biometric, optical, thermal, or electrical signals between one or more sensors and the interface target. In some embodiments, this can include optical windows, thermally conductive materials, electrical pads, or inductive couplings that align with complementary structures in a wearable device or the user's skin.

In an exemplary embodiment, coupling between the wearable device 200 and the biometric bridge system 100/500, and between the biometric bridge system 100/500 and the wearable device recharger 220, can be achieved through a variety of means including friction-fit, snap-fit, adhesive pads, mechanical latches, magnetic coupling, and other suitable ways of coupling, depending on implementation preferences and form factor considerations.

With regard to mechanical coupling, the design may utilize complementary grooves, ridges, dimples, or recessed engagement features to ensure secure registration and prevent lateral or rotational slippage during use. These structural features may be disposed on the coupling region 106, the bottom surface 118, or other regions of the enclosure 102 to align and mate precisely with the wearable device coupling area 204 or the recharger interface 222.

In some embodiments, magnetic coupling may be used either as the primary coupling mechanism or in combination with mechanical alignment features. One or more magnetic elements may be embedded within the enclosure 102, the wearable device 200, or the recharger 220, arranged to generate a magnetic attractive force that aligns the components and holds them in position during operation or charging. This approach enables tool-free attachment and detachment, improves ease of use, and ensures consistent positioning for sensing, alignment, and power transfer.

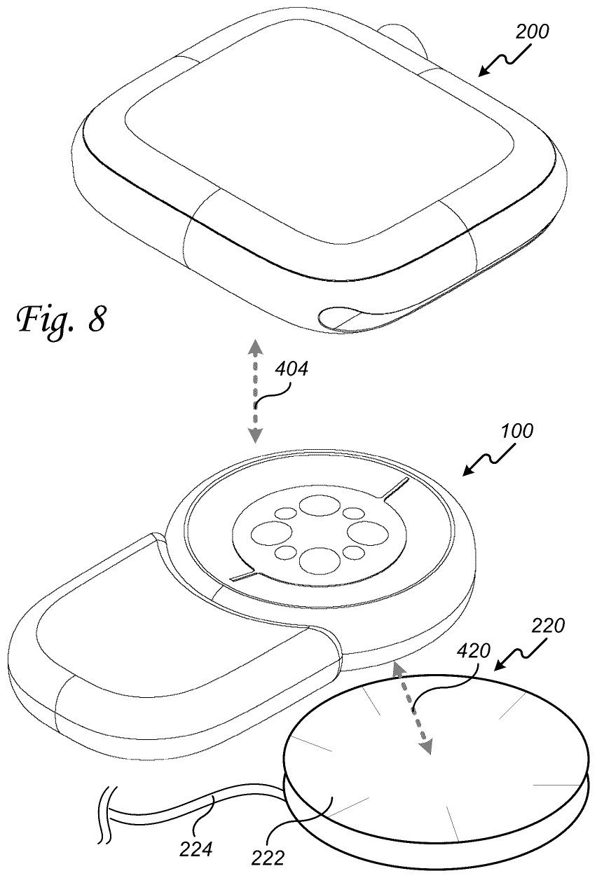

This same convex structure of the skin-contact region 120 is further configured to align 420 with a standard wearable device recharger 220 mating surface 222, enabling the system to support dual-sided docking—mating with the wearable device 200 on one side and the recharger 222 on the other, as better illustrated in at least FIG. 8. In operation, a power cord 224 can supply power to the wearable device recharger 220, which in turn can be used to recharge the wearable device 200, the biometric bridge system 100, or the combined assembly of the wearable device 200 coupled to the biometric bridge 100 at the same time.

The invention is not limited to any specific coupling technique or alignment orientation and supports a wide variety of implementation options across different mechanical, electrical, and ergonomic configurations.

Conventional smartwatches and fitness wearables typically require direct contact with the user's skin to perform sensing functions such as heart rate monitoring, blood oxygen measurement, skin temperature tracking, and echocardiogram (ECG) recording. These devices are designed with sensor arrays positioned on the back surface of the device, which must be in continuous proximity to the skin to function effectively.

However, existing smartwatches cannot be charged while worn, because the physical connection to a charging base or magnetic puck obstructs the sensor interface or requires removal of the device. As a result:
Users must sacrifice sensing capability during charging cycles, often disrupting sleep tracking or round-the-clock health monitoring;
There is no opportunity to enhance or expand sensing capabilities beyond what is pre-integrated into the wearable device;
Communication between the wearable device and cloud-connected or companion systems is disconnected during charging unless mediated by another device; and
Inaccessible or poorly aligned sensors during recharging lead to loss of biometric continuity.

Additionally, prior approach solutions do not allow for biometric sensing pass-through, sensor relaying, or sensor enhancement, nor do they support dual-sided mechanical alignment with both the wearable device and its native charger in a reversible manner.

The present biometric bridge system 100 solves the above limitations and others through a novel combination of mechanical, electrical, and data communication components integrated into a thin, ergonomically shaped insert that remains in contact with both the wearable device and the user's skin during operation.

Advantages of the present invention include:
Continuous Sensing During Charging: The biometric bridge system enables uninterrupted biometric signal acquisition using three operational pathways:
   Pass-through sensing 406 from the wearable device through the bridge to the user;
   Relay sensing 408 where bridge sensors acquire data and transmit it to the wearable device; and
   Enhanced sensing 410 where the bridge provides additional sensing capabilities not natively supported by the wearable device.
Ergonomic, Reversible Geometry: The biometric bridge includes a concave top surface and convex bottom surface, with alignment grooves, ridges, or dimples mirrored on both sides. This dual-mode compatibility allows the bridge to:
   Securely couple with the wearable device during use; and
   Be docked onto the wearable device's original charger without detaching the device from the bridge.
Expandable Functionality: Through its own embedded sensors and communication interface 508, the bridge can serve as a:
   Data augmentation platform, expanding the capabilities of the wearable device;
   Secure memory device, logging biometric data locally; and
   Bridge to the cloud, relaying data to computing devices 604 and remote servers 602 over a global network 600.
Power Sharing and Portability: The biometric bridge includes a rechargeable battery 526 and charging port 114, allowing it to:
   Recharge the wearable device;
   Recharge itself via external power (e.g., USB, solar, induction, other); and
   Serve as a backup power source for external devices such as smartphones.
Haptic Feedback Integration: A vibration module 522 within the bridge provides localized alerts for charging status, sensor misalignment, and health-based triggers.
Wearability and Usability: With its thumb tab 112, the device can be inserted and removed easily, even under tension from the wearable device band, making it suitable for continuous wear throughout the day and night.

Unlike conventional approaches, the present invention allows a user to wear a smartwatch during charging without compromising sensor functionality. It introduces a modular, interoperable interface that:
Integrates with existing wearable hardware without requiring redesign; Provides user-level and technician-level access to sensor and power systems; and
Acts as a multi-functional accessory, not just a passive pass-through element.

In the present invention, the term “biometric bridge” is intended to mean a thin, removable, ergonomically contoured electronic interface positioned between a wearable device and the skin of a user. The biometric bridge is configured to support and maintain biometric sensing, data communication, and power delivery operations while the wearable device remains worn. It forms a functional intermediary that physically and electrically couples the wearable device to the user's skin without disrupting wearable performance, even during charging cycles.

The biometric bridge can comprise:
A wearable-device-facing interface comprising a coupling region that physically mates with the underside of the wearable device;
A skin-facing sensor interface comprising one or more sensor contact points or proximity sensors;
A controller that manages sensing, power, and data operations;
A communication interface that allows the biometric bridge to exchange data with the wearable device, local computing devices, and global networks;
Optional structural components such as a thumb tab, alignment grooves/ridges, and concave/convex mating geometries;
A rechargeable power source and charging interface for charging the wearable device or acting as a standalone power supply;
Other features.

The biometric bridge enables multiple biometric sensing pathways:
Pass-through sensing pathway: Where the native sensors of the wearable device remain functional and operate through the biometric bridge via optical windows, thermal conductors, or electrical pass-through elements, allowing unimpeded signal access to the user's skin;
Relay sensing pathway: Where the biometric bridge includes its own sensors that capture biometric or environmental data and then relay that data to the wearable device via analog, digital, or wireless communication, effectively acting as an external sensor array; and
Enhanced sensing pathway: Where the biometric bridge includes sensors not present in the wearable device (e.g., blood pressure, non-invasive glucose, or bioimpedance). These enhanced readings may be:
   Transmitted directly to the wearable device for integration;
   Sent to an external computing device (e.g., smartphone or server); or
   Merged with data from the wearable device to expand the device's functional capabilities beyond its original design.

In all modes, the biometric bridge serves not just as a passive intermediary but as an active data and sensing enhancement module, allowing the wearable device to maintain, relay, or extend its sensing and data communication functions.

In the present invention, the term “biometric bridge system” is intended to mean an integrated assembly that includes the biometric bridge and its associated electronic, sensing, data communication, and power components. The biometric bridge system functions as a modular attachment compatible with wearable devices and designed to remain in contact with the user's skin during device operation.

The biometric bridge system can comprise:
The biometric bridge structure;
A controller that governs sensing logic, communication protocols, and power regulation;
One or more biometric or environmental sensors;
A communication interface supporting data exchange via Bluetooth®, NFC, USB, Wi-Fi, or other suitable protocols;
A power supply (e.g., rechargeable battery) and charging components;
Dual-sided physical geometry that allows coupling with both the wearable device and external recharging hardware; and
Other features.

The data communication functionality of the biometric bridge system enables:
Direct transmission of data to or from the wearable device;
Communication with computing devices such as smartphones, tablets, laptops, and desktop computers;
Wireless or wired access to a global communication network, such as the Internet, allowing cloud synchronization, remote monitoring, or telehealth integrations; and
Interoperability with remote data processing resources, including cloud-based analytics servers, electronic health record systems, and remote firmware update platforms.

By integrating pass-through, relay, and enhanced sensing pathways, the biometric bridge system provides data that augments and extends the capabilities of the wearable device, transforming it into a more advanced health, diagnostic, or connectivity tool than it would be when worn alone.

In the present invention, the term “wearable device” is intended to mean any electronic device designed to be worn on the body of a user and which includes one or more sensor arrays, communication interfaces, and rechargeable power components. A wearable device may include, but is not limited to:
Smartwatches, such as those worn on the wrist, including commercially available and proprietary devices;
Fitness trackers, health monitoring bands, or biosensor-equipped wearables;
Wearable medical monitoring equipment;
Devices including sensors for heart rate, oxygen saturation, ECG, body temperature, activity tracking, sleep monitoring, or communication; or
Any device that interfaces with the user's skin for sensing, power transfer, or interaction.

In the context of this invention, a wearable device comprises a wearable device coupling area, a sensor array, and optionally a recharging interface, each of which may be mechanically or electronically coupled with the biometric bridge or biometric bridge system.

In the present invention, the term “alignment structure” is intended to mean a mechanical geometry, such as a groove, ridge, dimple, or corresponding feature, disposed on one or more surfaces of the biometric bridge enclosure and configured to ensure precise positioning, stable mating, and repeatable orientation with a wearable device or a wearable device recharging system. Alignment structures may be complementary on opposite surfaces to enable reversible dual-side docking functionality.

In the present invention, the term “sensor interface” is intended to mean a physical, electrical, optical, thermal, or other communication region through which sensor signals are transferred. The term encompasses both the wearable-device-facing interface (receiving signals or aligning with device sensors) and the skin-facing interface (transmitting signals to or receiving signals from the user). A sensor interface may include optical windows, conductive surfaces, thermal bridges, electrode contact pads, or other features.

In the present invention, the term “global network” is intended to mean a communication infrastructure that facilitates data exchange beyond local or device-level protocols. A global network includes, but is not limited to, the Internet, cellular data networks, or satellite-based systems that enable the biometric bridge system to transmit or receive data from geographically remote computing resources or cloud servers.

In the present invention, the term “dual-mating functionality” is intended to mean the ability of the biometric bridge system to mechanically and functionally couple on one side with a wearable device, and on the opposite side with a wearable device charging dock. This functionality is enabled by mirrored or complementary structural alignment features that support consistent docking orientation and uninterrupted operational alignment across both use and recharge scenarios.

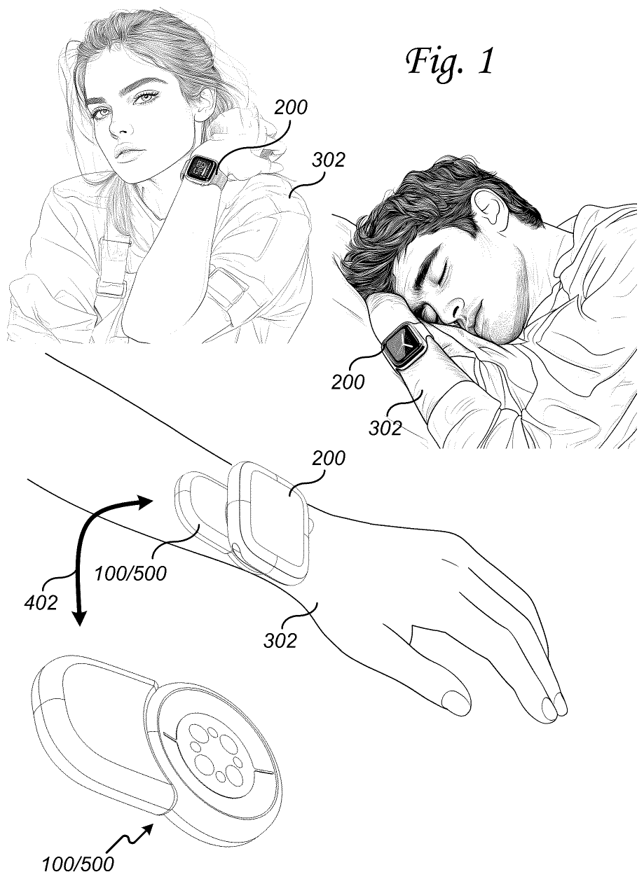

Turning now to the drawings in greater detail, it will be seen that in FIG. 1, there is illustrated one example of a biometric bridge system 100/500 inserted between a wearable device 200, such as a smartwatch, and the skin of a user 302. In an exemplary embodiment, the biometric bridge system 100/500 can be positioned beneath the wearable device 200 by a user 302 through a sliding motion, represented as motion 402, such that the biometric bridge system 100/500 is interposed between the device 200 and the user's skin 302 while the device remains on the wrist.

As shown in FIG. 1, multiple instances of user 302 are presented in the same illustration to show the progression of the insertion step. The wearable device 200 remains positioned on the forearm or wrist, while the user 302 slides the biometric bridge system 100/500 into place beneath it. The illustrated approach highlights how the system can be adopted without requiring substantial adjustment to the wearable device 200's orientation or band.

This insertion technique allows for integration with an already-worn wearable device 200 without requiring its removal. In practice, a user 302 can partially loosen or tilt the wearable device 200 and guide the biometric bridge system 100/500 beneath it by aligning the enclosure and gently sliding it into position along the wrist, thereby completing motion 402. Once inserted, the device 200 can be tightened against the biometric bridge system 100/500 to restore its secure fit. This process enables rapid placement with minimal disruption to the user's daily routine and does not require full disassembly of the wearable setup.

By enabling this seamless insertion (motion 402) and maintaining continuous sensor functionality, the biometric bridge system 100/500 differentiates itself from prior approaches that only support sensor contact in the absence of a charging interface or require device removal to initiate recharging. The ability to wear and charge a device 200 while preserving data continuity represents a significant advancement in both user convenience and device utility.

One key advantage of the present biometric bridge system 100/500, as illustrated in FIG. 1, is its ability to maintain sensor readings and data continuity even while the wearable device 200 is being charged or slightly repositioned. In prior approaches, users were forced to remove their smartwatches to charge them, resulting in interrupted biometric monitoring—particularly problematic during sleep tracking, when consistent heart rate, motion, and temperature data are especially valuable.

By contrast, the present system enables the user 302 to continue wearing the device 200 during both waking and sleeping hours, with the biometric bridge system 100/500 serving as a physical and functional intermediary that preserves sensor-to-skin contact through one or more sensing pathways, such as pass-through sensing 406, relay sensing 408, or enhanced sensing 410 (as described in later figures). This configuration can ensure uninterrupted data capture over prolonged periods, providing health platforms or analytics systems with a richer, more reliable stream of biometric inputs.

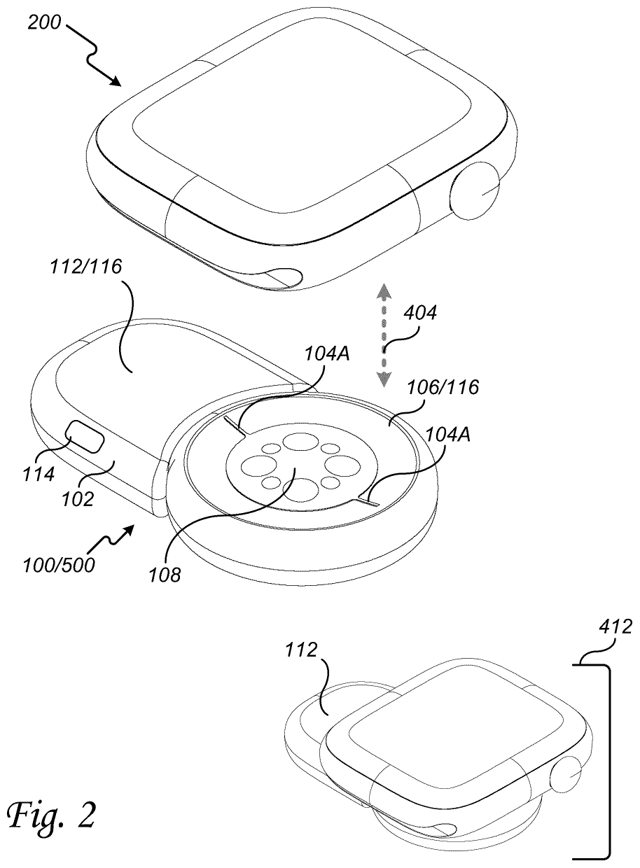

Referring to FIG. 2, there is illustrated one example of a biometric bridge system 100/500 aligning and coupling with a wearable device 200, where the top surface 116 of the biometric bridge system 100 and the wearable device 200 are visible. In an exemplary embodiment, the biometric bridge system 100 includes an enclosure 102 comprising a top surface 116 and a bottom surface 118, with an internal cavity housing a controller 500, sensors, a rechargeable battery 526, and associated interfaces.

The top surface 116 of the biometric bridge includes a coupling region 106 that physically interfaces with a wearable device coupling area 204 located on the underside of the wearable device 200. To achieve precise and secure alignment, the coupling region 106 incorporates one or more alignment structures 104, such as grooves, ridges, or dimples, specifically configured to engage with complementary features of the wearable device 200. In this figure, the alignment features are located on the top surface 104A, ensuring proper seating and alignment during motion 404, which corresponds to the motion of coupling and decoupling the watch from the biometric bridge 100.

Also visible in this figure is the thumb tab 112, which protrudes laterally from the enclosure 102 and allows the user to easily position, insert, or remove the biometric bridge 100 during the coupling process. This is particularly advantageous when the wearable device band is partially secured around the wrist, reducing the need for device removal or readjustment.

In an exemplary embodiment, the rechargeable battery 526, controller 500, and associated components such as communication interface 508 and sensor suite 512 may be contained entirely within the main body of the enclosure 102, including the coupling region 106 that aligns with the wearable device 200. In such configurations, the thumb tab 112 may simply function as a grip element for ergonomic placement and removal, without housing any critical components.

Alternatively, in other embodiments, the thumb tab 112 may optionally house portions of the controller, the battery, or one or more sensors. The invention is not limited to any specific internal arrangement of components within the enclosure 102 or the thumb tab 112. The battery, sensing elements, and charging interfaces may be co-located beneath the wearable device, distributed across the enclosure, or contained within a single central housing, depending on design preferences, device geometry, or manufacturing constraints.

This flexibility in internal layout allows the biometric bridge system 100/500 to accommodate various form factors and electrical configurations without departing from the scope of the invention.

FIG. 2 also shows the charging port 114, which allows for wired recharging of the rechargeable battery 526 within the enclosure 102. Charging the biometric bridge may also occur wirelessly, such as by induction charging or other suitable methods. The battery may also supply current to the wearable device 200 during operation.

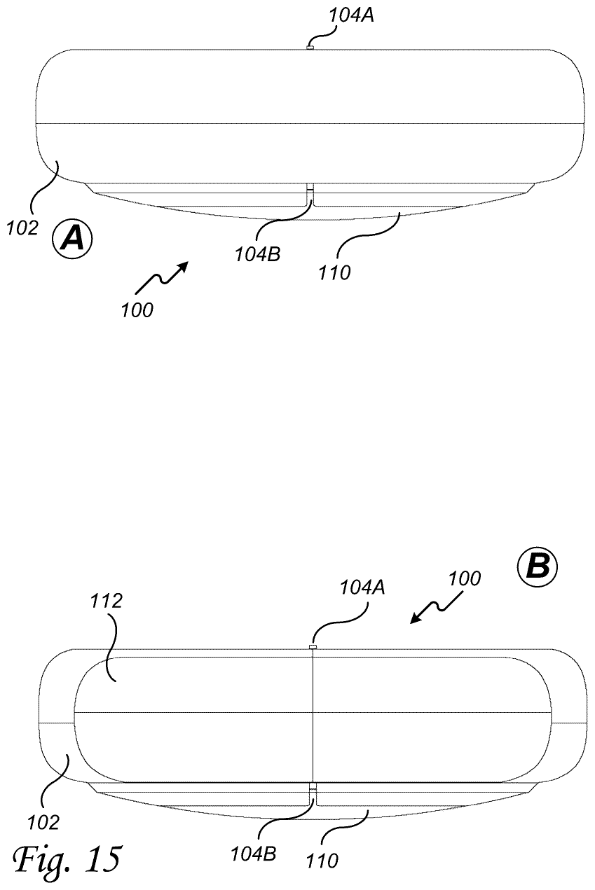

Of particular importance in this embodiment is the structural configuration that enables dual-mating functionality, as detailed in the invention disclosure. The top surface alignment features 104A and their bottom-side counterparts 104B (better illustrated in at least FIG. 15) are inverted—that is, if the top surface 104A includes a ridge, the bottom surface 104B includes a complementary groove, and vice versa. This mirrored design allows the biometric bridge system 100 to mate with the wearable device 200 during use, and then later dock with the wearable device's original charging base when the device is removed. This geometry supports reversibility, compatibility, and simplified transitions between use and recharging modes.

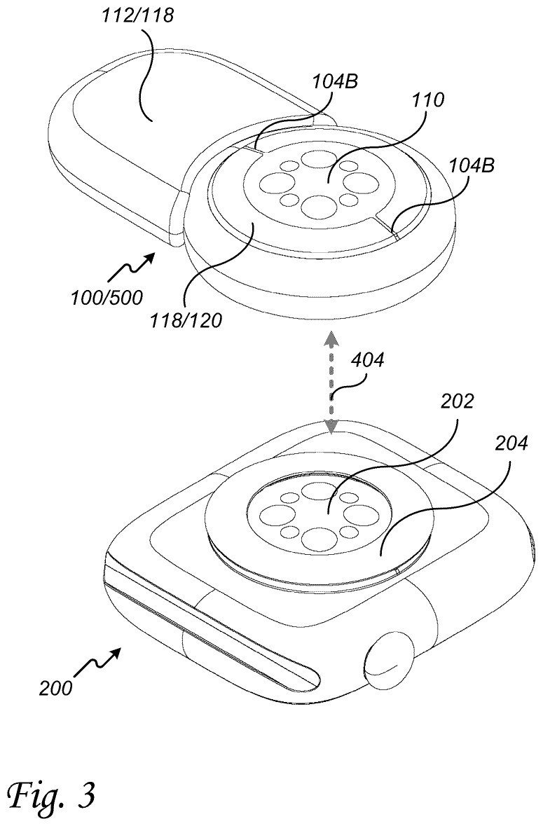

Better illustrated in at least FIG. 3, the bottom surface 118 includes a skin contact region 120, which is configured to remain in direct contact with the user's skin 302 during use. In addition to hosting skin-contact sensors 110, the skin contact region 120 can also be configured to electrically couple with a wearable device charger when the biometric bridge is flipped or repositioned for charging. This structural and electrical dual-functionality of the skin contact region 120 distinguishes the present invention from prior approaches, which do not contemplate such reversibility or dynamic role-switching between skin interface and charging interface.

The stable coupling shown in this figure is further depicted in the coupled view 412, where the wearable device 200 is seated onto the biometric bridge system 100. This figure reinforces the precision fit achieved by the structural features described above and sets the foundation for ongoing sensor interface 108 alignment and data continuity through one or more of pass-through sensing 406, relay sensing 408, or enhanced sensing 410.

These features not only improve reliability and user experience but also establish a platform for continuous sensing, modular expansion, and seamless integration with existing wearable ecosystems, thereby addressing shortcomings in prior approaches that require full removal of the wearable device for charging or do not support sensor extension or physical interoperability.

Referring to FIG. 3, there is illustrated one example of a biometric bridge system 100/500 aligning and coupling with a wearable device 200, where the bottom surface 118 of the biometric bridge and the underside of the wearable device 200 are visible. In an exemplary embodiment, this view reveals the mechanical, sensing, and ergonomic alignment features that allow the biometric bridge system 100/500 to interoperate seamlessly with the wearable device 200.

The bottom surface 118 of the biometric bridge system 100 includes a skin contact region 120, which is configured to maintain continuous contact with the user's skin 302. This region can incorporate one or more skin-contact sensors 110, including optical windows, electrical sensing pads, thermally conductive materials, or bioimpedance electrodes. These components can support multiple operational modes:
Pass-through sensing 406, wherein sensor signals from the wearable device sensor array 202 are transmitted through the biometric bridge to wearable interface 108 and onto the user's skin via the skin contact region 120;
Relay sensing 408, wherein the biometric bridge 100 includes embedded sensors that capture biometric data and transmit it to the wearable device 200; and
Enhanced sensing 410, wherein the biometric bridge provides sensing capabilities not present in the wearable device, such as non-invasive glucose or blood pressure monitoring.

The wearable device coupling area 204, visible from this view, aligns with the coupling region 106 on the top surface 116 of the biometric bridge. The coupling region 106 includes one or more alignment features 104, shown here as part of top surface alignment structures 104A. These features—grooves, ridges, or dimples—are configured to engage complementary features of the wearable device coupling area 204 to promote stable mechanical positioning and precision sensor alignment.

The action of joining or separating the wearable device 200 from the biometric bridge system 100 is represented by 404, which corresponds to the motion of coupling and decoupling the watch from the biometric bridge 100. This motion can be performed by the user 302 using the thumb tab 112, which extends outward from the enclosure 102. The thumb tab 112 enables easy positioning of the biometric bridge system 100 beneath the wearable device 200 and supports its removal without fully loosening or removing the watch band. The design supports one-handed use and improves usability during daily routines or overnight wear.

The enclosure 102 is further defined by a design-conscious form that enhances both functionality and visual integration. In particular, the end portion of the enclosure 102 that includes the coupling region 106 is dimensioned and contoured to match the shape and edge geometry of the wearable device 200 and in particular the geometry of the wearable device coupling area 204. This ensures a smooth, low-profile transition between the two devices, such that the biometric bridge system 100 appears to blend into the underside of the wearable device when viewed from the top or side. This approach reduces visual distraction, preserves aesthetic continuity, and maintains a clean device silhouette, even when the biometric bridge system 100 is actively coupled.

The thumb tab 112 is joined to the same coupling end of the enclosure 102 and is proportioned such that it remains visible and grippable by the user 302, without extending excessively beyond the device footprint. The result is an integrated physical form that enables:
Reliable sensor coupling;
Easy user operation;
Minimal aesthetic disruption; and
Other features.

In addition, the skin contact region 120 is convex in curvature to follow the anatomical contour of the user's wrist or forearm, which supports secure sensor placement and user comfort over long durations. That same surface can also be configured to couple with a wearable device charger, allowing the biometric bridge system 100 to recharge its internal rechargeable battery 526 using the device's native recharging system. This dual-function design of the bottom surface supports alignment compatibility and dual-mating functionality, as further defined in Section 16 of the invention disclosure.

Together, the structural elements shown in FIG. 3—including the enclosure shape, the coupling geometry, the thumb tab 112, the alignment structures 104A, and the dual-role contact region 120—enable a mechanically secure, aesthetically pleasing, and functionally advanced interface between the biometric bridge system 100 and the wearable device 200. These features promote continuous biometric sensing, simplify recharging, and elevate the overall user experience in ways not achievable by typical add-on accessories or rigid adapter modules.

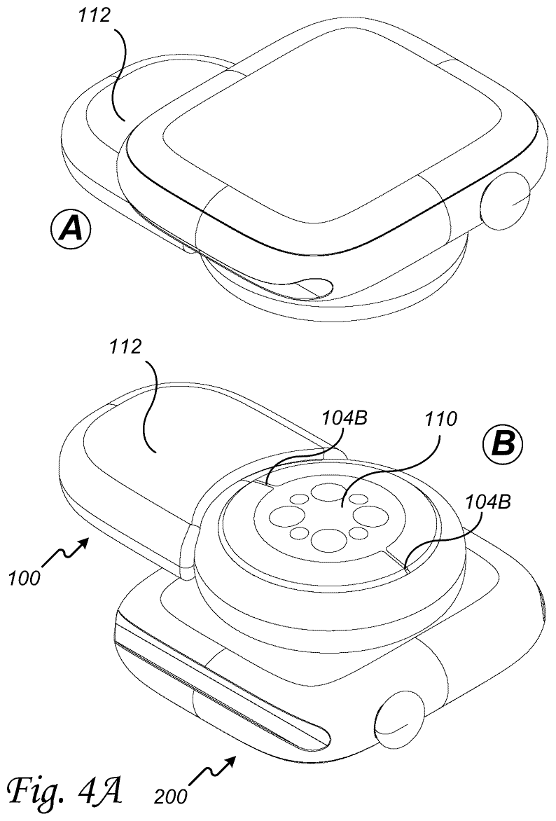

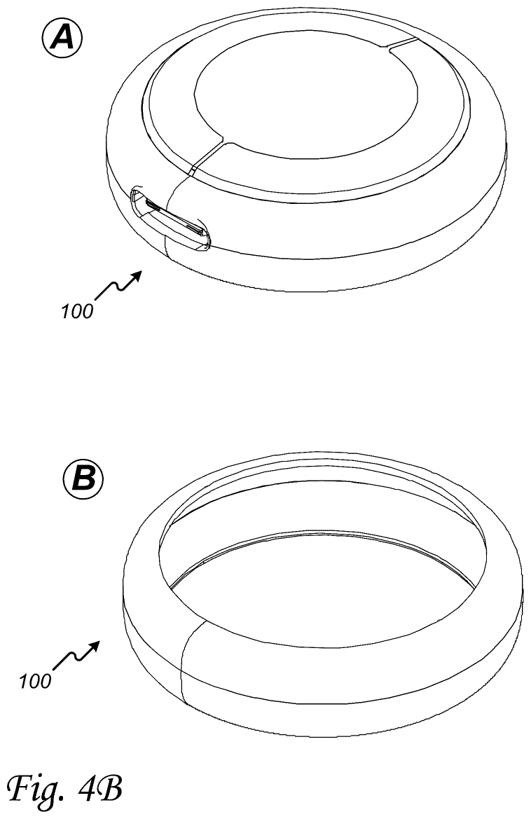

Referring to FIG. 4A, there is illustrated one example of a biometric bridge system 100/500 coupled to a wearable device 200, shown from two distinct perspectives: a top surface perspective view and a bottom surface perspective view. In an exemplary embodiment, this figure highlights the full integration of the biometric bridge system 100 with the wearable device 200, both structurally and functionally, with attention to the device's physical form, sensor alignment, and coupling fidelity.

In the top surface perspective view, reference ‘A’, the top surface 116 of the biometric bridge enclosure 102 is visible as it mates flush against the wearable device coupling area 204. The interface between the coupling region 106 and the underside of the wearable device 200 is shown as a unified surface, where the alignment structures 104A—comprising grooves, ridges, or dimples—are engaged with complementary mating features of the wearable device. This alignment ensures stable mechanical coupling and precise positioning of the sensor interface 108 relative to the wearable device sensor array 202.

The alignment of the top surface 116 with the edges and footprint of the wearable device 200 demonstrates the minimal protrusion and cohesive form factor achieved by the invention. This configuration enhances wearability by maintaining a low profile and ensuring that the system does not interfere with clothing, wrist movement, or user activity. The top view reinforces how the design of the coupling region 106 and surrounding enclosure is optimized to match the aesthetic and mechanical footprint of the wearable device 200.

The bottom surface perspective view, reference ‘B’, provides a clear view of the bottom surface 118 of the biometric bridge system 100, including the skin contact region 120. This surface is configured to maintain uninterrupted contact with the user's skin 302, facilitating biometric data collection via one or more skin-contact sensors 110 embedded within or flush-mounted to the surface. In this view, the curvature of the skin contact region 120 is apparent, following the natural convex contour of the human wrist to improve comfort and ensure consistent sensor-to-skin engagement across a range of wrist anatomies.

Also visible from the bottom is the thumb tab 112, which extends laterally from the coupling end of the biometric bridge 100. From this vantage point, the user-operable position of the thumb tab 112 is evident, allowing a user 302 to apply light upward or downward pressure to initiate motion 404, the coupling or decoupling of the biometric bridge 100 from the wearable device 200. This ergonomic integration of the thumb tab enables single-handed removal or repositioning, without interfering with the appearance or operation of the wearable device.

The dual-perspective layout of FIG. 4A uniquely showcases the dual-surface architecture of the biometric bridge system. On the top side, the enclosure 102 acts as a structural and sensing intermediary with the wearable device 200. On the bottom side, the enclosure provides a biometric interface and, when reversed or docked, can also serve as a charging surface, allowing the rechargeable battery 526 to be replenished through the same region that contacts the user's skin.

Referring to FIG. 4B, there is illustrated one example of a biometric bridge system 100/500 configured in alternative geometries, including a disc-shaped embodiment (reference A) and a ring-shaped embodiment (reference B). These configurations demonstrate the versatility of the invention's form factor and reinforce that the biometric bridge system is not limited to any particular enclosure shape or external contour, such as the elongated design with a thumb tab shown in other figures.

In an exemplary embodiment, reference A depicts a disc-shaped biometric bridge, wherein the enclosure 102 has a generally circular footprint with a top surface 116 and a bottom surface 118. The coupling region 106 remains centrally located on the top surface and is configured to engage the underside of a wearable device 200, such as a smartwatch. The bottom surface 118 includes the skin-contact region 120, which may contain one or more sensor interfaces 110 as described elsewhere in the application.

This disc configuration eliminates the thumb tab 112, offering a symmetric, minimalistic profile that may be more aesthetically compatible with circular smartwatches or wearable devices with centrally located charging and sensing elements. The absence of protruding geometry can improve comfort for the user 302, reduce snagging during wear, and offer uniform sensor pressure distribution against the skin.

In an alternative embodiment shown in reference B, the biometric bridge is configured as a hollow ring, where the enclosure 102 forms a toroidal or donut-like shape. This ring configuration may surround a central open space and can be designed to align with central optical or electrical sensor arrays of the wearable device. The top surface 116 of the ring structure still includes a coupling region 106 that mates with the wearable device coupling area 204, while the bottom surface 118 includes a distributed or annular skin-contact interface 120 that may support sensing around the perimeter of the ring.

This ring-shaped embodiment can offer several unique advantages, including:
Unobstructed access to centrally located sensors or electrodes on the wearable device that must contact the skin directly.
Weight reduction, since material in the center can be omitted.
Ventilation and comfort improvements, particularly during sleep or high-activity use.
Potential integration with wristbands or straps, where the bridge may straddle or align with existing wearable hardware.

Both of these alternative geometries (disc and ring) demonstrate that the biometric bridge system 100/500 is structurally and functionally adaptable. The core system components—such as the controller 500, sensor interfaces 110, rechargeable battery 526, and alignment features 104A/104B—can be repositioned or redistributed within various enclosure geometries without deviating from the scope of the invention.

These embodiments provide enhanced flexibility in terms of industrial design, user comfort, mechanical compatibility, and market-specific customization, making the biometric bridge system suitable for a wide range of wearable device platforms and user preferences.

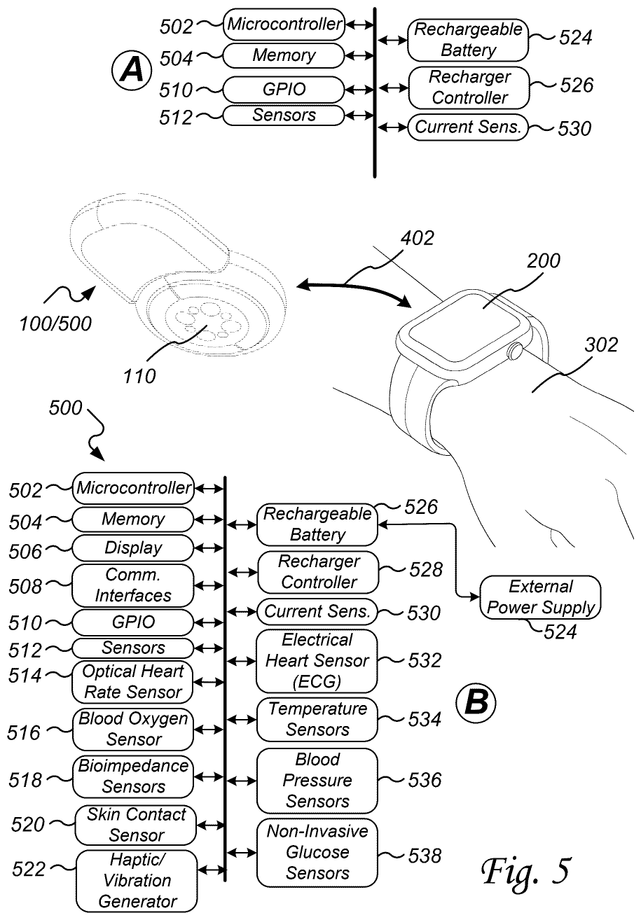

Referring to FIG. 5, there is illustrated examples of a system block diagram of a controller 500 integrated into the biometric bridge system 100/500. The biometric bridge system includes an enclosure 102, which houses the controller 500 and various electrical and sensing components. The combination of the enclosure 102 and the controller 500 is collectively referred to as the biometric bridge 100/500.

In one configuration, referred to as reference ‘A’, the controller 500 can comprise a simplified architecture, including one or more of the following: microcontroller 502, memory 504, GPIO 510, sensors 512, external power supply 524, rechargeable battery 526, recharge controller 528, and current sensor 530. This configuration can be suitable for basic sensing, pass-through or relay modes, and power management.

In a more comprehensive configuration, referred to as reference ‘B’, the controller 500 can include one or more of the features described herein, including: microcontroller 502, memory 504, display 506, communication interface 508, GPIO 510, sensors 512, optical heart rate sensor 514, blood oxygen sensor 516, bioimpedance sensors 518, skin contact sensors 520, haptic vibration generator 522, rechargeable battery 526, recharge controller 528, current sensor 530, electrical heart sensor 532, temperature sensors 534, blood pressure sensors 536, and non-invasive glucose sensors 538. An external power supply 524 can be used to recharge the rechargeable battery 526, or used in other ways. Additionally, the external power supply 524 can be the wearable device recharger 220.

In an exemplary embodiment, the biometric bridge system 100/500 further comprises a battery management system (BMS) integrated within the controller 500, designed to monitor and control the performance, safety, and longevity of the rechargeable battery 526. In the present invention, the term “battery management system” is intended to mean a subsystem of the controller 500 that is configured to manage the charging, discharging, thermal regulation, and protection of the rechargeable battery under varying operational conditions.

The battery management system can comprises several components of controller 500, including but not limited to:

The recharge controller 528, which governs the charging process by controlling current and voltage levels delivered from the external power supply 524 to the rechargeable battery 526. The recharge controller 528 may support various charging protocols such as constant current (CC), constant voltage (CV), or pulse charging, and may be configured to switch between modes based on battery state and environmental conditions.

The current sensor 530, which continuously measures the current entering and exiting the battery during both charging and discharging cycles. The current sensor data is used to track power usage trends, estimate remaining battery capacity, and detect anomalies such as excessive current draw or leakage conditions.

The temperature sensors 534, which may include internal and/or ambient thermal sensors to ensure that charging and discharging processes are suspended or adjusted if the temperature exceeds safe thresholds. This thermal regulation prevents overheating and reduces risk of battery degradation or thermal runaway.

The microcontroller 502, which aggregates sensor input, applies logic and safety thresholds, and executes firmware instructions stored in memory 504 to make battery management decisions in real time. The microcontroller may also log charge/discharge cycles and perform predictive analytics to extend battery life.

The battery management system may also include fault detection mechanisms, such as undervoltage lockout, overvoltage protection, short circuit detection, and reverse polarity safeguards, all of which contribute to device safety and compliance with applicable battery safety standards.

Through the integration of these components, the battery management system not only protects the rechargeable battery 526 but also ensures that power delivery to the wearable device 200 and internal subsystems remains stable, efficient, and safe throughout various modes of operation—including sensing, wireless transmission, and idle standby.

The controller 500 can also be configured to operate as an Internet of Things (IoT) and web-enabled device. In the context of the present invention, this means the controller 500 can connect via the communication interface 508 to external computing devices or remote data processing resources over a global network 600, such as the Internet, which is a global network 600. This functionality enables features such as remote diagnostics, over-the-air firmware updates, biometric data upload to cloud storage, integration with third-party health applications, and real-time health monitoring. The web-enabled IoT capability of the controller 500 transforms the biometric bridge system 100/500 into a dynamic, intelligent, and connected accessory that expands the functionality and interactivity of the wearable device 200.

Through the combination of these components, the controller 500 enables the biometric bridge 100/500 to function as a smart, connected, and autonomous sensing and power management accessory for a wearable device 200. The IoT functionality of the controller allows the system to operate as a web-enabled node that supports real-time monitoring, cloud integration, and remote diagnostics.

In one exemplary embodiment, the controller 500 comprises a microcontroller 502, which is operationally related to each of the following components:

The memory 504 can be used to store firmware, sensor calibration parameters, biometric logs, or user-specific configuration data. It may include volatile or non-volatile memory types such as RAM, ROM, flash memory, or EEPROM.

The display 506 can be a status display integrated into the biometric bridge 100/500 and may include light emitting diode (LED), liquid crystal display (LCD), or other types of visual indicators. It can be used to display battery status, connection state, or user alerts.

The communication interface 508 allows the controller 500 to data communicate with the wearable device 200, external computing devices such as smartphones, tablets, or PCs, and remote data processing resources over a network. Supported protocols may include Bluetooth, Wi-Fi, USB, NFC, or other suitable standards. This configuration enables the biometric bridge system 100/500 to operate as an Internet of Things (IoT) device that is web-enabled.

The general purpose input and outputs (GPIO) 510 provide flexible digital and analog signal interfaces to other components or peripherals within the enclosure 102.

A variety of different sensors 512 can be supported, each connected to the controller 500. These sensors may measure biometric, environmental, or positional data.

An optical heart rate sensor 514 can be integrated to measure the heart rate of the user 302. This sensor may be used during pass-through, relay, or enhanced sensing modes.

A blood oxygen sensor 516 may also be included to measure the blood oxygen level of the user 302. This can provide real-time SpO₂ tracking and contribute to assessments of respiratory and circulatory health, especially in sleep monitoring, fitness tracking, or medical evaluation contexts. These sensors can be used in enhanced sensing modes.

Bioimpedance sensors 518 may be included for capturing impedance-based biometric data, such as hydration levels or tissue characterization.

Skin contact sensors 520 can determine whether the biometric bridge 100 and/or the wearable device 200 are actively worn by the user 302. These sensors help reduce false readings and optimize power usage by determining when the system is in contact with the user.

A haptic vibration generator 522 can be included to generate tactile feedback to the user 302. Multiple operational modes can be supported. In one mode, the biometric bridge system 100/500 can be mechanically coupled to the wearable device 200 such that vibrations generated by the wearable device are physically conducted through the biometric bridge to the user's skin. In another mode, the vibration generator 522 can sense vibration from the wearable device 200 and, in response, activate its own vibration to create a user-detectable tactile event. Whether through direct mechanical coupling or sensed-response generation, the haptic vibration generator 522 can also be used independently to provide alerts or notifications originating from the biometric bridge system 100/500. These alerts may include biometric bridge low rechargeable battery warnings, indication that the wearable device 200 charging is complete, sensor misalignment, or other user or system-related notifications.

The external power supply 524 can be a transformer, solar panel, portable battery, wearable device recharger 220, or other rechargeable power source. It is temporarily connected to the controller 500 of the biometric bridge 100/500 for the purpose of recharging the rechargeable battery 526 and for other operational needs, such as powering the controller during updates or diagnostics. In certain embodiments, the external power supply 524 may include or be integrated with the recharging hardware normally used for the wearable device 200, allowing for shared or interchangeable charging interfaces.

In an exemplary embodiment, when the wearable device 200 is not being worn by the user 302, the wearable device 200 can be mechanically coupled to the biometric bridge system 100/500, forming an integrated assembly. The combined assembly can then be coupled to an external power supply 524, which may include the wearable device charging station or other suitable recharging interface. In this configuration, both the wearable device 200 and the biometric bridge system 100/500 are aligned to receive charging current simultaneously. The mirrored or complementary alignment features 104A and 104B on the top and bottom surfaces of the enclosure 102 ensure proper orientation and docking with the charging system. This arrangement enables dual-device charging using a single power supply without requiring decoupling or the use of multiple adapters, preserving sensor alignment, simplifying user workflow, and supporting continuous device readiness.

The rechargeable battery 526 is used to power the biometric bridge 100/500 and may also provide charging current to the wearable device 200.

The recharge controller 528 manages the charge and discharge cycles of the rechargeable battery 526. It ensures safe operation, controls charge timing, and may optimize power distribution between the biometric bridge and wearable device.

The current sensor 530 measures the current flow into and out of the rechargeable battery 526. This data can be used to determine charging state, track power usage, and detect anomalies.

An electrical heart sensor (ECG) 532 is provided to measure the user's 302 electrocardiogram. It may operate independently or in coordination with other sensors.

Temperature sensors 534 can measure skin, ambient, or internal device temperatures. This data can be used for user health tracking or thermal regulation of the device.

Blood pressure sensors 536 may be included to monitor the user's 302 blood pressure. These sensors can be used in enhanced sensing modes.

Non-invasive glucose sensors 538 allow for estimation of the user's 302 blood glucose level without skin puncture or fluid extraction. These sensors expand the sensing capabilities of the wearable ecosystem.

With regards to skin-contact sensing, in an exemplary embodiment, the skin-contact sensor interface 110 of the biometric bridge system 100/500 includes an optical window aligned with a photoplethysmographic (PPG) sensor of the wearable device 200. The optical window may be formed from a transparent or optically transmissive material such as sapphire glass, polycarbonate, acrylic, or other low-scatter polymers, and is positioned within the bottom surface 118 of the enclosure 102, specifically within the skin-contact region 120. The optical window is optically aligned with the photodetector and light emitter elements of the PPG sensor located on the underside of the wearable device coupling area 204. In this configuration, the sensor interface 108 enables pass-through sensing 406, allowing light emitted by the wearable device's PPG sensor (typically red, infrared, or green wavelengths) to pass through the window, reflect from the user's skin, and return through the same optical path for detection. This alignment allows for uninterrupted heart rate monitoring, heart rate variability analysis, and peripheral blood flow estimation, even when the skin bridge system is present between the wearable and the skin.

In an exemplary embodiment, the skin-contact sensor interface 110 includes a light sensor specifically designed to estimate or monitor blood sugar levels using spectroscopy. This sensor may utilize near-infrared (NIR), mid-infrared (MIR), or visible light-based optical spectroscopy to perform non-invasive glucose measurement. The sensor may include one or more tunable or fixed-wavelength light emitters and one or more photodetectors configured to measure skin reflectance, transmission, or absorption spectra in targeted spectral bands that correlate with glucose levels in interstitial fluids or capillaries. The light sensor may be embedded within or adjacent to the skin-contact region 120 of the enclosure 102, and operatively coupled to the controller 500, which may include signal processing algorithms or memory 504 with calibration models to interpret the detected optical signals. The glucose sensor may function independently of the wearable device or in tandem with its onboard systems, and can operate as part of an enhanced sensing mode 410, providing the user with continuous, non-invasive blood glucose monitoring capabilities through the biometric bridge.

In an exemplary embodiment, the skin-contact sensor interface 110 comprises one or more sensors selected from an electrical heart sensor, a temperature sensor, or a bioimpedance sensor, all operatively coupled to the controller 500 within the skin bridge system 100/500.

The electrical heart sensor may include electrocardiographic (ECG) electrodes embedded within the skin-contact region 120 and spaced to establish a measurable electrical potential across the user's skin. These electrodes are configured to detect cardiac electrical activity, enabling the system to generate ECG waveforms for heart rhythm analysis or arrhythmia detection.

The temperature sensor may comprise contact thermistors, resistance temperature detectors (RTDs), or infrared thermopile elements, capable of measuring skin surface temperature or ambient temperature near the body. This information can support circadian rhythm tracking, fever detection, or temperature-compensated biometric analysis.

The bioimpedance sensor may include a combination of current source electrodes and voltage measurement electrodes, used to assess electrical impedance across body tissues. This data can be used to estimate hydration status, body composition, respiration rate, or other physiological markers.

These sensors may operate individually or in combination, depending on use case and sensing mode, and can support relay sensing 408 or enhanced sensing 410 based on whether the wearable device 200 incorporates corresponding sensing capabilities. In all cases, sensor data is captured by the biometric bridge and processed or forwarded by the controller 500 for use by local or remote computing systems.

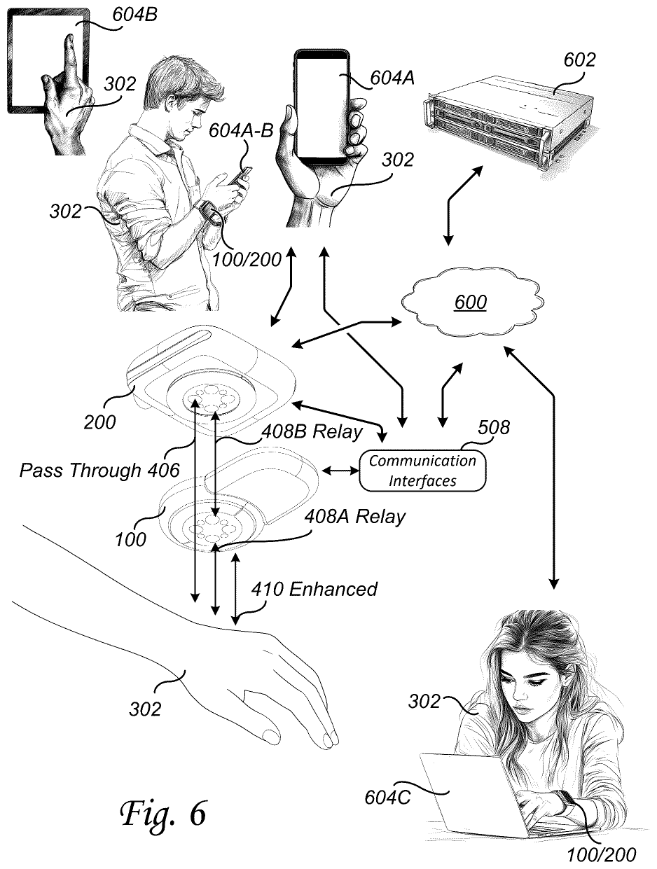

Referring to FIG. 6, there is illustrated one example of a network diagram, highlighting how the biometric bridge system 100/500 interacts with various connected devices and systems to support advanced sensing modes and dynamic data communication. In an exemplary embodiment, the controller 500 within the biometric bridge system 100/500 is configured to interface with a wearable device 200, local computing devices 604A, 604B, and 604C, and a remote data processing resource 602, also referred to as a server 602, using one or more communication pathways facilitated by the communication interface 508.

The communication interface 508 can include wireless protocols such as Bluetooth, Wi-Fi, NFC, cellular standards, as well as wired communication protocols such as USB, or other suitable standards and/or protocols. Through this interface, the controller 500 is capable of:
Exchanging data with the wearable device 200, including sensor data, control signals, and power coordination commands;
Communicating with computing devices 604A, 604B, and 604C, which can include smartphones, tablets, laptops, and desktop computers for localized user interaction, data review, or device configuration;
Establishing secure links to a remote server 602, enabling integration with cloud-based services, over-the-air firmware updates, long-term biometric data storage, or healthcare provider networks; and
Other capabilities.

This multi-level connectivity architecture positions the biometric bridge 100/500 as a web-enabled Internet of Things (IoT) node that extends the reach, intelligence, and utility of the wearable device ecosystem.

FIG. 6 also illustrates three key modes of biometric sensing integration, each of which differentiates the biometric bridge system 100/500 and expands the functional scope of the wearable device 200:
Pass-Through Sensing 406: In pass-through sensing mode 406, sensor signals from the wearable device 200 are transmitted through the biometric bridge 100/500 and delivered directly from the skin of the user 302 via properly aligned optical, thermal, or electrical pathways. This is enabled by structural elements like sensor interface 108, skin-contact region 120, and conductive or transparent channels within enclosure 102.
This configuration allows the user to charge the wearable device or reorient it without interrupting the device's native sensing operation—an advantage over prior approaches where charging physically disables sensing due to the required detachment from the user.
Relay Sensing 408A and 408B: in relay sensing 408a-b, the sensing process is divided into two linked actions:
   408A represents the capture of biometric signals (e.g., temperature, heart rate, ECG, other) by the biometric bridge system 100/500 using its own sensor suite 512, such as optical heart rate sensor 514, ECG 532, bioimpedance sensors 518, or other sensors; and
   408B represents the transmission of the captured data from the biometric bridge controller 500 to the wearable device 200. This can occur by way of the communication interface 508, using either wired or wireless communication methods such as Bluetooth, Wi-Fi, or USB, or by direct electrical coupling. Additionally, the transmission may be performed by generating a signal—analog, digital, optical, or other—that stimulates the wearable device's own sensor interface. In this approach, the wearable device interprets the biometric bridge-generated signal as though it originated from its native sensors, enabling seamless data handoff without requiring changes to the wearable device's firmware or sensor architecture.
   Relay sensing 408A-B offers a powerful advantage when wearable device sensors are obstructed or inactive. In such cases, the biometric bridge system 100 can function as an intermediary sensing module that supplements or replaces the data stream without requiring modifications to the wearable device firmware or hardware.
Enhanced Sensing 410: Enhanced sensing 410 refers to sensing capabilities that do not exist within the wearable device 200 but are enabled by the biometric bridge system 100/500.

Such enhanced sensing 410 can include advanced biometric modalities such as:
Blood pressure sensing 536;
Non-invasive glucose monitoring 538;
Blood oxygen measurement 516;
Cortisol measurement;
Skin temperature and ambient sensing 534; and
Other types and/or kinds of sensing.
Captured data from these sensors can include:
Transmitted to the wearable device 200 for display or further processing;
Routed to computing devices 604A-C for real-time visualization or alerting;
Synced with server 602 for analytics, cloud storage, or healthcare integration; or
Other used in other ways, as may be required and/or desired in a particular embodiment.

This mode turns the biometric bridge into a data augmentation platform, transforming a basic wearable into a more comprehensive health-tracking system. The result is a modular enhancement that scales the wearable device's utility without requiring hardware redesign.

Collectively, the architecture shown in FIG. 6 illustrates how the biometric bridge system 100/500 enables:
Uninterrupted sensing during device recharging;
Continuous health monitoring during sleep or activity;
Flexible local and global data communication; and
Sensing feature expansion, effectively upgrading the user's wearable device with capabilities not included at the time of purchase.

This represents a significant differentiation from prior solutions, which typically lack the ability to both passively support existing sensors and actively introduce new sensing and communication pathways into the wearable ecosystem.

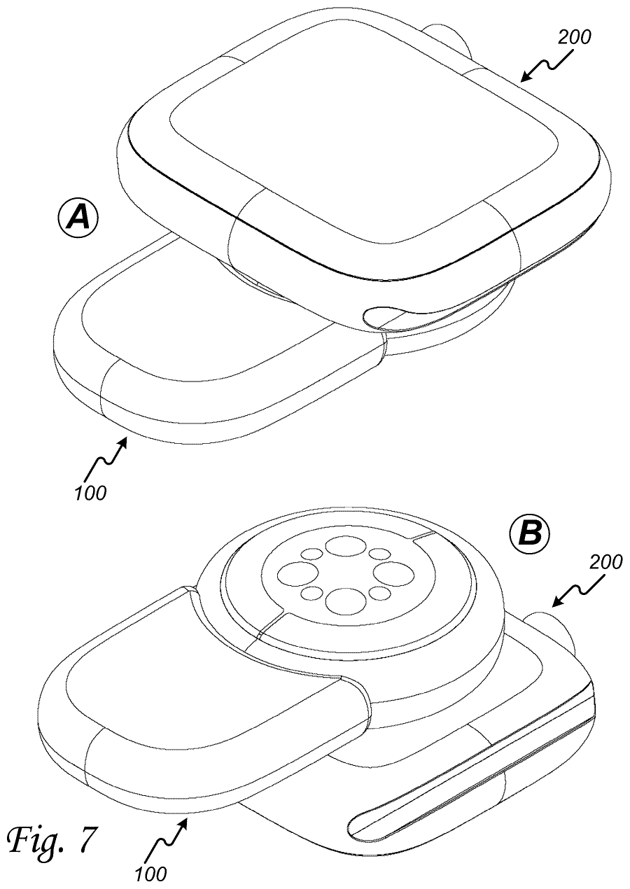

Referring to FIG. 7, there is illustrated one example of a top perspective view, reference ‘A’ and a bottom perspective view, reference ‘B’ of the wearable device 200 coupled to the biometric bridge system 100/500. In an exemplary embodiment, this figure demonstrates the full integration of the two devices in both form and function, showcasing how the biometric bridge 100/500 operates in harmony with the wearable device 200 without altering its outward appearance or usability.

In reference ‘A’ (top view), the top-facing surface of the wearable device 200 is shown along with the top surface 116 of the biometric bridge enclosure 102 beneath it. The coupling region 106 of the biometric bridge system 100/500 aligns flush with the wearable device coupling area 204, enabling stable mechanical engagement. The alignment features 104A, comprising one or more grooves, ridges, or dimples, are not overtly visible in this view but are embedded within the interface region to ensure proper alignment and coupling.

This top view reinforces the aesthetic advantages of the present invention: the biometric bridge system 100/500 adds functionality without compromising the engineered visual profile of the wearable device 200. Enclosure 102 is designed with matching curvature and width, blending into the device footprint so as not to create excess bulk or uneven surfaces. This promotes comfort and discreet wearability across waking and sleeping contexts.

Visible from the top is the thumb tab 112, extending laterally from the edge of the enclosure 102 near the coupling region 106. Its location and contour are ergonomically optimized for gripping by the user 302, allowing for easy engagement or disengagement of the biometric bridge system 100/500 during motion 402 (insertion) or motion 404 (coupling/decoupling). This top view illustrates how the thumb tab 112 integrates visually with the rest of the system while remaining accessible.

In reference ‘B’ (bottom view), the bottom surface 118 of the biometric bridge system 100/500 is visible, including the skin contact region 120. This region features a convex shape designed to match the natural curvature of the user's wrist or forearm. The skin contact region 120 may include embedded skin-contact sensors 110, including those responsible for pass-through sensing 406, relay sensing 408, or enhanced sensing 410 as previously described. Depending on configuration, this region can include sensors such as optical heart rate sensor 514, bioimpedance sensors 518, skin contact sensors 520, temperature sensors 534, or ECG sensors 532, among others.

The bottom view also shows how the biometric bridge 100/500 can serve as a charging interface. The skin contact region 120 can be configured to reversibly couple with the wearable device's native charging dock, using bottom-side alignment features 104B that mirror the geometry of 104A. For example, if the top alignment surface includes ridges, the bottom may include grooves of matching shape. This dual-mating geometry enables the user to flip or reorient the biometric bridge system for recharging without detaching it from the wearable device 200, supporting on-body charging or standalone battery replenishment.

The bottom perspective reinforces how the biometric bridge system 100/500 serves as a bridge not only between the wearable device 200 and the user 302, but also between sensing, power, and data ecosystems. The enclosed controller 500 can remain operational in either orientation, allowing data acquisition, power delivery, or cloud synchronization to continue while the device is charging or removed.

Taken together, references ‘A’ and ‘B’ in FIG. 7 illustrate the mechanical integration, aesthetic continuity, ergonomic accessibility, and bidirectional interfacing capabilities of the biometric bridge system 100/500. These views distinguish the present invention from conventional charging adapters or band inserts by demonstrating a highly engineered coupling mechanism that supports multi-surface interaction, advanced sensing, and modular use—all within a form factor that complements the original design of the wearable device.

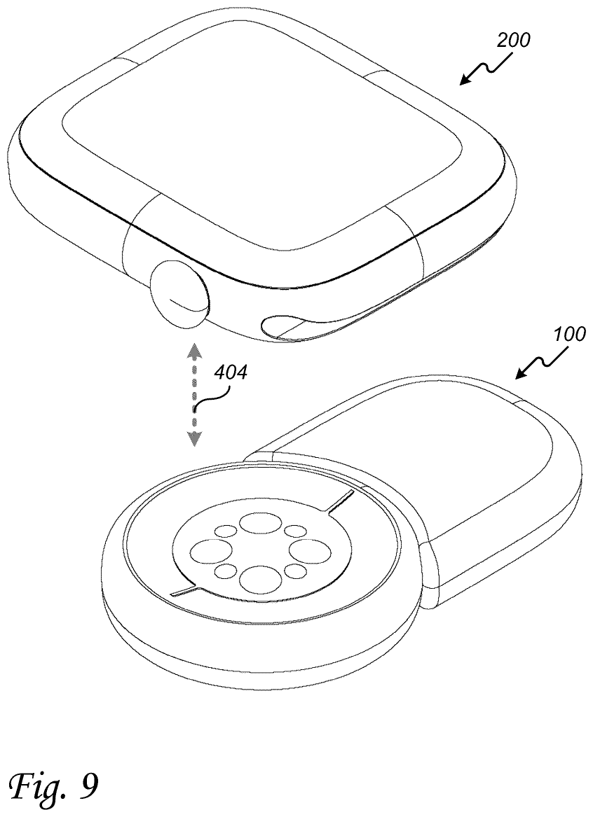

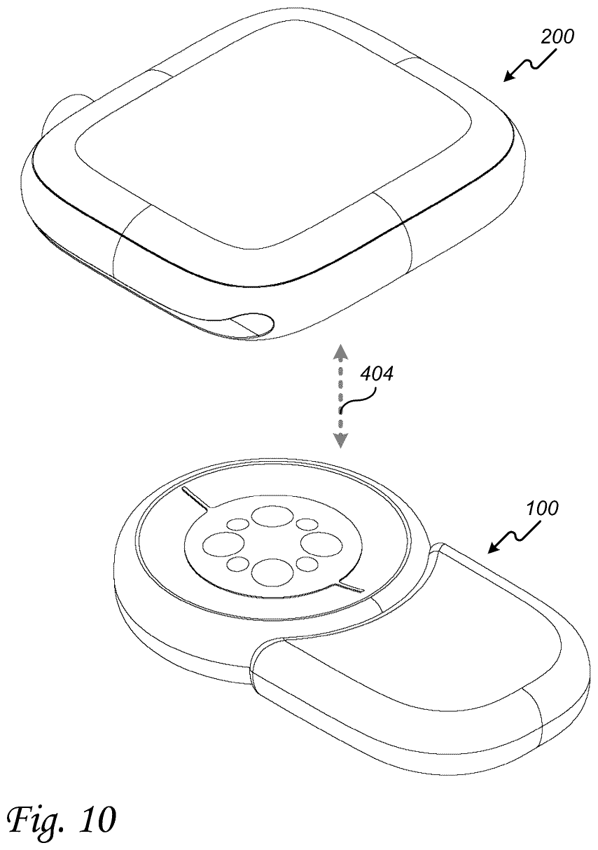

Referring to FIGS. 8 through 10, there are illustrated examples of top perspective views of the uncoupled wearable device 200 and the biometric bridge system 100/500. In an exemplary embodiment, these figures show the two components in an uncoupled, side-by-side configuration, highlighting the alignment-ready interface geometry and the complementary form factors designed to enable secure, low-profile coupling.

The top surface 116 of the biometric bridge enclosure 102 is visible, including the coupling region 106 which is shaped to match the contour and footprint of the wearable device coupling area 204. While the alignment features 104A (grooves, ridges, or dimples) may not be visible from these angles, their location and purpose are structurally supported by the interface surfaces shown.

Also apparent in these views is the thumb tab 112, which extends from the edge of the biometric bridge enclosure near the coupling region. Its location and contour are designed for user accessibility during insertion or removal, aiding the coupling 404 and sliding 402 motions required to align and seat the biometric bridge beneath the wearable device 200.

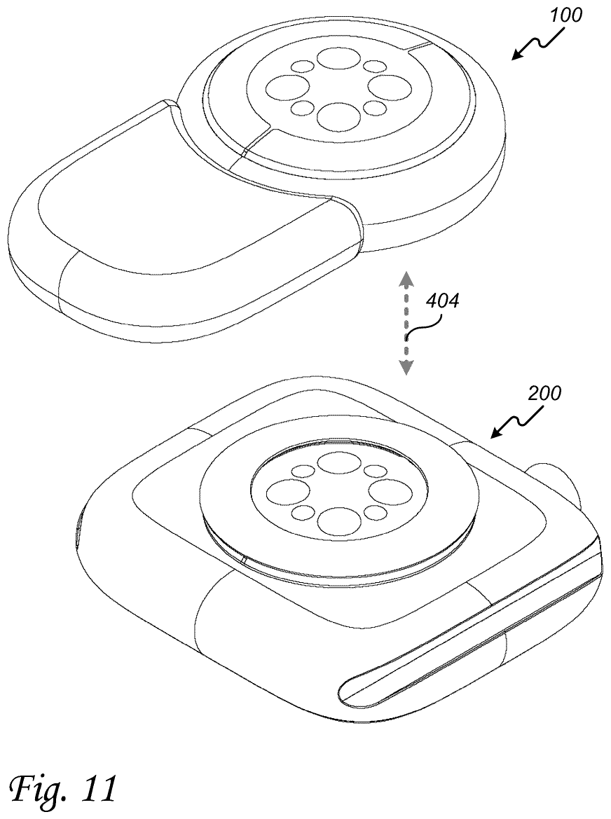

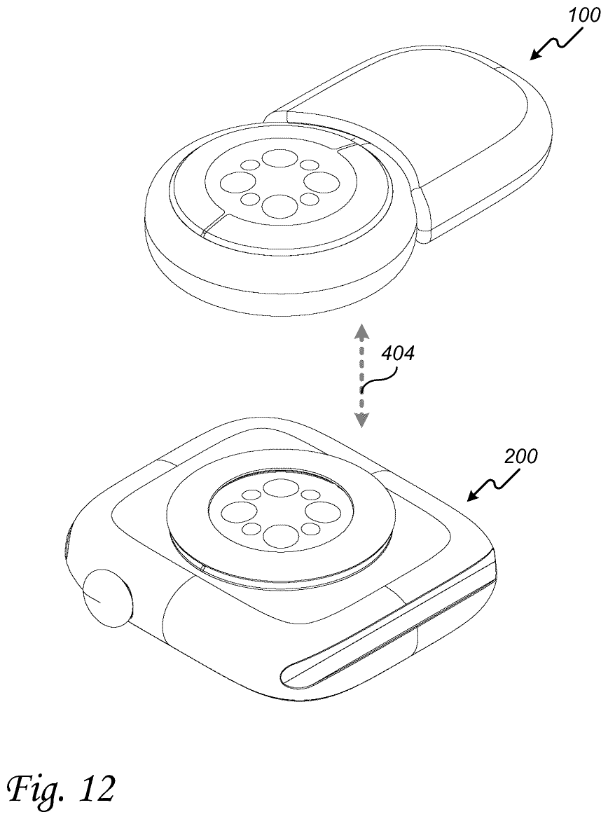

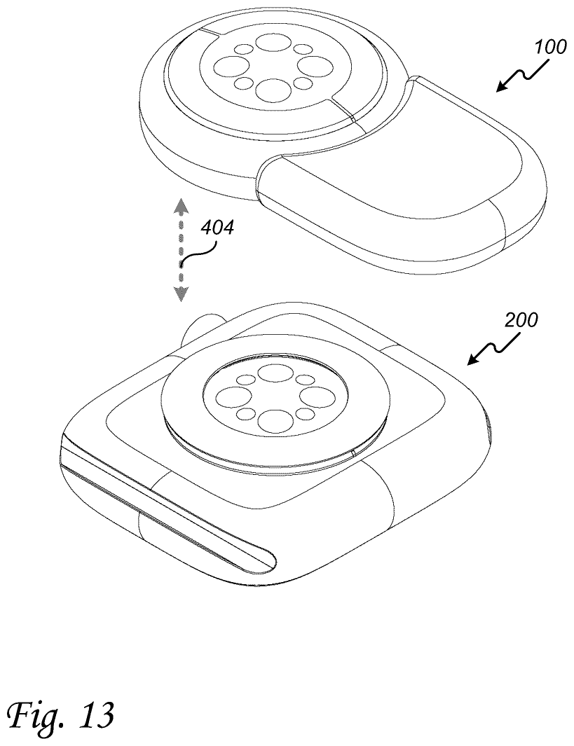

Referring to FIGS. 11 through 13, there are illustrated examples of bottom perspective views of the uncoupled wearable device 200 and the biometric bridge system 100/500. In an exemplary embodiment, these views show the underside of each component, highlighting the skin contact region 120 of the biometric bridge system 100/500 and the wearable device coupling area 204 of the wearable device 200, in an uncoupled state.

The bottom surface 118 of the biometric bridge enclosure 102 is visible, revealing the convex curvature of the skin contact region 120, which is ergonomically designed to conform to the wrist or forearm of the user 302. This region can host various skin-contact sensors 110, including biometric sensing components such as optical heart rate sensors 514, temperature sensors 534, ECG contacts 532, and others.

From this view, the spatial alignment between the biometric bridge and the wearable device becomes more apparent. The bottom perspective makes it easier to observe the geometry intended for dual-surface compatibility, where the bottom surface 104B of the biometric bridge 100/500 includes alignment features (not explicitly labeled in this view) that are complementary to those on the top surface 104A. These mirrored structures facilitate reversible docking—either with the wearable device during use, or with a wearable device recharger 220 when charging.

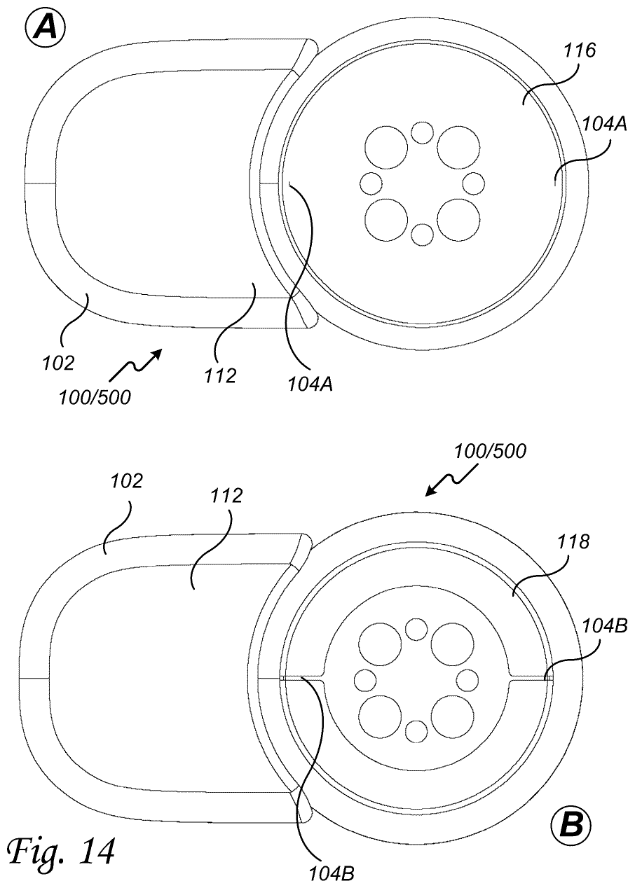

Referring to FIG. 14, there is illustrated one example of a top view, reference ‘A’, and a bottom view, reference ‘B’ of the biometric bridge system 100/500. In an exemplary embodiment, these views provide a complete look at the form and function of the enclosure 102 from both sides, revealing the integrated design features that support coupling, sensing, and reversible charging.

In reference ‘A’ (top view), the top surface 116 of the enclosure 102 is shown, including the coupling region 106 that interfaces with the wearable device 200 during use. This region includes one or more alignment features 104A, such as grooves, ridges, or dimples, which are dimensioned and positioned to ensure precise mechanical engagement with the wearable device coupling area 204. The thumb tab 112 is also visible, extending from the end of the enclosure adjacent the coupling region 106, and is ergonomically shaped for manual gripping to facilitate insertion or removal of the biometric bridge system.

In reference ‘B’ (bottom view), the bottom surface 118 is visible, prominently featuring the skin contact region 120, which has a convex curvature designed to match the contour of the user's wrist or forearm. This surface is configured to remain in full contact with the user's skin during use and can include a variety of skin-contact sensors 110, such as optical sensors, ECG electrodes, temperature sensors, and bioimpedance electrodes. These components support pass-through sensing 406, relay sensing 408, and enhanced sensing 410 modes, depending on the system configuration.

The bottom view also reveals that the alignment features 104B on the bottom surface are inverted counterparts to the alignment features 104A on the top surface. That is, if the top surface includes a ridge, the bottom surface includes a corresponding groove, and vice versa. This mirrored geometry allows the biometric bridge system 100/500 to dock 420 into a wearable device recharger 220 in the same manner it mates with the wearable device 200, enabling dual-mating functionality for seamless transitions between use and recharging.

Referring to FIG. 15, there is illustrated one example of a front view, reference ‘A’, and a back view, reference ‘B’, of the biometric bridge system 100/500. In an exemplary embodiment, these views emphasize the lateral profile, dimensional proportions, and structural balance of the enclosure 102, offering visual confirmation of the system's compact form factor and ergonomic design.

In reference ‘A’ (front view), the top surface 116 and the thumb tab 112 are prominently visible. The thumb tab 112 is located adjacent to the coupling region 106, and extends laterally outward with a gentle arc or flange. This positioning ensures that the user 302 can easily grip and manipulate the biometric bridge system 100/500 during insertion (motion 402) and decoupling (motion 404). The view also emphasizes the flat, low-profile nature of the top surface, designed to sit flush against the wearable device coupling area 204.

The front view further illustrates the width symmetry of the enclosure 102, which is engineered to match the width of the wearable device 200 for seamless integration without overhang or mismatch. This contributes to user comfort and enhances the visual continuity of the wearable device when the biometric bridge is in place.

In reference ‘B’ (back view), the bottom surface 118 is shown with the skin contact region 120 visible. The back view highlights the convex curvature of the skin contact region 120, which is shaped to contour to the surface of the user's wrist or forearm. This ergonomic geometry is designed to maintain even pressure distribution and continuous sensor contact during motion, regardless of wrist orientation or activity.

The back view also provides a clear profile of the vertical thickness of the enclosure 102, which is minimized to reduce the overall height of the wearable device and biometric bridge assembly. Despite housing key components such as the controller 500, rechargeable battery 526, sensor suite 512, and optional vibration module 522, the biometric bridge maintains a thin and balanced structure optimized for all-day wear.

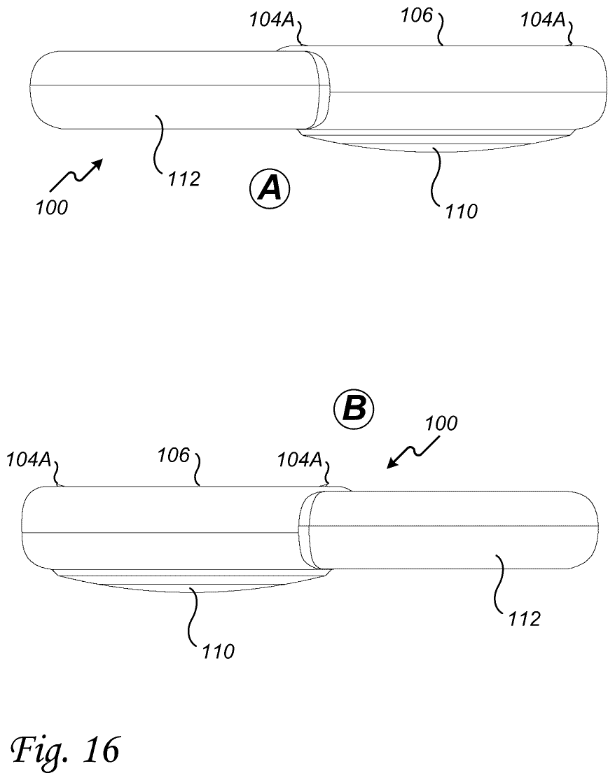

Referring to FIG. 16, there is illustrated one example of a left view, reference ‘A’ and a right view, reference ‘B’ of the biometric bridge system 100/500. In an exemplary embodiment, these side views further define the three-dimensional profile of the enclosure 102, with emphasis on the contoured structure, interface geometry, and accessibility of user-operable features.

In reference ‘A’ (left view), the thumb tab 112 is prominently shown as it extends outward from the coupling end of the biometric bridge. The side view reveals the tapered thickness of the thumb tab 112 relative to the main body of the enclosure 102, highlighting its low-profile integration that maintains aesthetic cohesion while offering a functional gripping point for the user 302. This tab facilitates the insertion (motion 402) and decoupling (motion 404) of the biometric bridge system 100/500 from beneath the wearable device 200.

The left view also illustrates the slight curvature or convex contour of the bottom surface 118, particularly within the skin contact region 120, designed to ergonomically match the user's wrist while maintaining even contact pressure for biometric sensing. The vertical profile also reveals the gradual elevation from the tab end to the rear edge, accommodating the internal architecture of the controller 500, sensor suite 512, and rechargeable battery 526 without introducing discomfort or bulk.

In reference ‘B’ (right view), the opposite side of the biometric bridge enclosure 102 is visible. While symmetrical in overall form, this view further confirms the enclosure's balanced shape, clean lines, and structural uniformity, which ensure proper alignment with the wearable device 200 and consistent coupling on either side. From this angle, the viewer can also infer the mirror-image design of the alignment features 104A and 104B located along the top and bottom surfaces, enabling dual-mating functionality with both the wearable device 200 and its recharger 220.

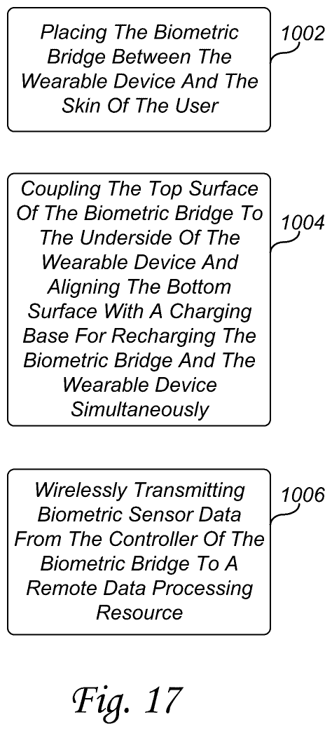

Referring to FIG. 17, there are illustrated examples of methods of using a biometric bridge system 100/500, demonstrating its integration with a wearable device 200, a user 302, and remote data infrastructure. In an exemplary embodiment, the steps highlight the operational modes of the biometric bridge system from on-body sensing to off-body recharging and remote communication.

In step 1002, the method includes placing the biometric bridge system 100/500 between the wearable device 200 and the skin of the user 302. The placement is performed such that:
The sensor interface 108 between the top surface 116 of the biometric bridge and the underside (coupling area 204) of the wearable device is properly aligned;
The skin-contact region 120 on the bottom surface 118 of the enclosure 102 is securely in contact with the user's skin.

This alignment ensures that biometric signals can flow through or originate from the biometric bridge system and be detected by either the biometric bridge itself or the wearable device using any of the three modes of sensing: pass-through sensing 406, relay sensing 408, or enhanced sensing 410. The ergonomic shape of the enclosure 102, combined with the thumb tab 112, allows the user to easily insert and seat the biometric bridge without removing the wearable device.

In step 1004, the method includes coupling the top surface 116 of the biometric bridge system 100/500 to the underside of the wearable device 200 and aligning the bottom surface 118 with a charging base. This configuration supports recharging both the biometric bridge system 100/500 and the wearable device 200 simultaneously when the system is not being worn by the user 302. Thanks to the mirror-image alignment features 104A and 104B located on the top and bottom surfaces of the enclosure 102, the same form-fitting geometry used to engage the wearable device during on-body use can be inverted and applied to a wearable device charging base. These alignment features—whether grooves, ridges, or dimples—ensure a stable mechanical connection and promote accurate electrical or inductive coupling for efficient power transfer.

This dual-mating functionality allows the user to transition the system from a worn state to a recharging state without detaching or disassembling the components. The geometry enables the biometric bridge system to remain mechanically integrated with the wearable device while simultaneously interfacing with the existing charging platform, eliminating the need for additional adapters, connectors, or dedicated charging cradles.

In step 1006, the method includes wirelessly transmitting biometric sensor data from the controller 500 of the biometric bridge system to a remote data processing resource 602 over a global network.

This transmission is performed via the communication interface 508, which may support wireless protocols such as Wi-Fi, Bluetooth, NFC, or cellular communication. The remote data processing resource 602, also referred to as a server, can receive biometric data collected by the sensor suite 512, such as heart rate, skin temperature, blood oxygen, blood glucose, and other user health metrics.

This cloud communication step transforms the biometric bridge system 100/500 into a web-enabled, IoT health accessory, capable of:
Performing long-term biometric logging;
Supporting remote diagnostics or alerts;
Aggregating sensor data for later syncing with the wearable device 200;
Passing data through to a healthcare provider, smartphone, or application in real time; or
Execute other features.

The capabilities of the present invention can be implemented in software, firmware, hardware, or some combination thereof.

As one example, one or more aspects of the present invention can be included in an article of manufacture (e.g., one or more computer program products) having, for instance, computer usable media. The media has embodied therein, for instance, computer readable program code means for providing and facilitating the capabilities of the present invention. The article of manufacture can be included as a part of a computer system or sold separately.

Additionally, at least one program storage device readable by a machine, tangibly embodying at least one program of instructions executable by the machine to perform the capabilities of the present invention can be provided.

The flow diagrams depicted herein are just examples. There may be many variations to these diagrams or the steps (or operations) described therein without departing from the spirit of the invention. For instance, the steps may be performed in a differing order, or steps may be added, deleted, or modified. All of these variations are considered a part of the claimed invention.

While the preferred embodiment of the invention has been described, it will be understood that those skilled in the art, both now and in the future, may make various improvements and enhancements which fall within the scope of the claims which follow. These claims should be construed to maintain the proper protection for the invention first described.

What is claimed is:

1. A biometric bridge system configured to be positioned between a wearable device and the skin of a user, the system comprising:
an enclosure comprising a top surface and a bottom surface;
a coupling region on the top surface configured to engage a coupling area of the wearable device;
a first sensor interface on the top surface configured to communicate with a wearable device sensor interface; and
a second sensor interface on the bottom surface configured to contact the skin of the user;
wherein the first sensor interface and the second sensor interface are positioned and configured to enable sensing communication between the wearable device and the user through the biometric bridge.

2. The biometric bridge system of claim 1, further comprising a thumb tab integrally formed with the enclosure and positioned adjacent to the coupling region.

3. The biometric bridge system of claim 1, further comprising:
a controller housed within the enclosure and operatively coupled to the first and second sensor interfaces;
wherein the controller is configured to enable one or more of:
pass-through sensing, wherein the biometric bridge facilitates transmission of sensing signals from the wearable device to the user and return signals from the user to the wearable device through the biometric bridge without altering the signal content;
relay sensing, wherein the biometric bridge is configured to detect biometric signals from the user and transmit corresponding data signals to the wearable device, and to receive sensor-related signals from the wearable device for processing or response by the biometric bridge; or
enhanced sensing, wherein the biometric bridge includes one or more sensors configured to measure biometric parameters that are not sensed by the wearable device, and to generate corresponding data signals independently of the wearable device.

4. The biometric bridge system of claim 1, wherein the second sensor interface includes an optical window aligned with a photoplethysmographic sensor of the wearable device.

5. The biometric bridge system of claim 1, wherein the second sensor interface comprises one or more of an electrical heart sensor, a light sensor, a temperature sensor, or a bioimpedance sensor.

6. The biometric bridge system of claim 1, further comprising a rechargeable battery disposed within the enclosure and configured to recharge the wearable device.

7. The biometric bridge system of claim 2, further comprising a rechargeable battery disposed within the thumb tab and configured to recharge the wearable device.

8. The biometric bridge system of claim 1, wherein the controller comprises a microcontroller, memory, and a communication interface; and
wherein the communication interface comprises one or more of Bluetooth, near field communications (NFC), Wi-Fi, universal serial bus (USB), wireless, or wired communication connections.

9. The biometric bridge system of claim 1, wherein the second sensor interface includes a spectroscopy-based light sensor configured to measure blood glucose levels.

10. The biometric bridge system of claim 1, wherein the second sensor interface comprises a thermally conductive insert configured to pass heat from the user's skin to the wearable device.

11. A biometric bridge system configured to be positioned between a wearable device and the skin of a user, the system comprising:
an enclosure comprising a top surface and a bottom surface;
a sensor interface on the bottom surface configured to contact the skin of the user; and
a rechargeable battery disposed within the enclosure and configured to recharge the wearable device;
a coupling region formed on the top surface, the coupling region comprising one or more first alignment structures selected from magnets, grooves, ridges, or dimples; and
one or more second alignment structures disposed on the bottom surface of the enclosure, wherein the second alignment structures are complementary to the first alignment structures;
wherein when the wearable device is removed, the biometric bridge is alignable and mateable with a wearable device recharging system using the second alignment structures.

12. The biometric bridge system of claim 11, wherein the top surface comprises a concave coupling region configured to mate with a convex underside of the wearable device.

13. The biometric bridge system of claim 11, wherein the bottom surface comprises a convex sensor interface configured to conform to a user's skin and align with a charging base.

14. The biometric bridge system of claim 11, wherein the bottom surface conforms to a curvature compatible with a concave docking area of the wearable device recharging system.

15. A biometric bridge system configured to be positioned between a wearable device and the skin of a user, the system comprising:
an enclosure comprising a top surface and a bottom surface;
a coupling region configured to interface with the wearable device;
a rechargeable battery disposed within the enclosure and configured to recharge the wearable device, the rechargeable battery being monitored by a battery management system configured to track battery performance during recharging;
a controller housed within the enclosure, the controller comprising a microcontroller, memory, and a communication interface;
one or more biometric sensors operatively coupled to the controller;
wherein the communication interface is configured to transmit sensor data to one or more of:
the wearable device;
a computing device; or
a remote data processing resource over a global network.

16. The biometric bridge system of claim 15, wherein the memory is configured to store biometric data received from the wearable device or the one or more biometric sensors.

17. The biometric bridge system of claim 15, wherein the communication interface supports operation as a USB thumb drive for manual data transport.

18. The biometric bridge system of claim 15, further comprising a vibration module configured to generate alerts to the user.

19. The biometric bridge system of claim 15, wherein the controller is configured to aggregate biometric data from both the biometric bridge and the wearable device for unified data processing and analysis.

20. A method of using the biometric bridge system of claim 15, the method comprising the step of:
wirelessly transmitting biometric sensor data from the controller to a remote data processing resource over a global network.
